(19) United States
(12) Patent Application Publication
(10) Pub. No.: US 2013/0224141 A1
(43) Pub. Date: Aug. 29, 2013

(54) METHOD FOR STRAIGHTENING HAIR

(71) Applicants: Scott Robson, Babington (GB); Cheryl Anne Taylor, Bebington (GB)

(72) Inventors: Scott Robson, Babington (GB); Cheryl Anne Taylor, Bebington (GB)

(21) Appl. No.: 13/823,164

(22) PCT Filed: Sep. 12, 2011
(86) PCT No.: PCT/EP11/65746

§ 371 Date: May 14, 2013

(30) Foreign Application Priority Data
Sep. 24, 2010 (EP) 10179169.7

(51) Int. Cl. A61K 8/67; A61Q 5/04

(52) U.S. Cl. CPC A61K 8/678; A61Q 5/04; A61K 8/676
USPC 424/70.2

(57) ABSTRACT

A method of straightening hair comprising the step of applying to the hair a composition comprising at least 2 wt % of the total composition of vitamin C and vitamin E.

The invention relates to a method of straightening the hair.

Straight hair is seen by many consumers as desirable. Straightening products can be products that permanently straighten the hair by a chemical process or products that aid hair straightening with the use of straightening irons.

The use of ascorbic acid as a neutralising formulation for hair has been disclosed in U.S. Pat. No. 5,051,252.

WO 2005/025524 discloses a method of straightening hair with compositions comprising an alpha-hydroxy acid and a non-thio based reducing agent.

The present invention relates to effective straightening products. A further advantage of the present invention is that hair treated with these products has enhanced benefits on re-wash. That is not only do these products aid hair straightening on initial application, hair remains straight after the hair is washed and re-styled.

The present invention relates to a method of straightening hair comprising the step of applying to the hair a composition comprising at least 2 wt % of the total composition of vitamin C and vitamin E.

The total level of vitamin C and vitamin E present in the total formulation is greater than 2 wt %, preferably greater than 3.5 wt %, more preferably greater than 5 wt %. To obtain good sensory properties such as good hair feel and smoothness of the hair it is preferable if the total level of vitamin C and vitamin E present in the total formulation is less than 20 wt %, more preferably less than 15 wt %, most preferably 10 wt % or less.

It is preferable if the composition comprises at least 1 wt % vitamin C, more preferably at least 3 wt % vitamin C, most preferably at least 5 wt % vitamin C.

It is preferable if the composition comprises at least 0.5 wt % vitamin E, more preferably at least 1 wt % vitamin E, most preferably at least 2 wt % vitamin E.

It is preferable if the weight of vitamin C to vitamin E is greater than 1:1. More preferably the weight of vitamin C to vitamin E is greater than 3:2.

It is preferable if the weight of vitamin C to vitamin E is less than 5:1.

It is advantageous if the composition further comprises an α-hydroxy acid acids and salts thereof, of particular interest are bis (α-hydroxy acids) and/or their salts. α-Hydroxy acids which are of most importance for use with the present invention are citric acid and tartaric acid and/or their salts.

Compositions of the present invention are formulated into hair care compositions, especially products with hair straightening claims.

It is preferred if the products are left on hair after application and not immediately washed off (within 60 minutes of application). Such products are called “leave on” formulations.

Preferred product forms are leave on formulations such as gels, mousses, sprays, creams and aerosols. Creams and mousses are particularly preferred.

It is preferable if the pH at 30° C. of the composition is from 2.5 to 9, more preferably above 4, most preferably from 5 to 8.

Hair care compositions of the present invention can comprise a carrier, or a mixture of such carriers, which are suitable for application to the hair. The carriers are present at from about 0.5% to about 99.5%, preferably from about 5.0% to about 99.5%, more preferably from about 10.0% to about 98.0%, of the composition. As used herein, the phrase “suitable for application to hair” means that the carrier does not damage or negatively affect the aesthetics of hair or cause irritation to the underlying skin.

Compositions according to the invention comprise a buffer or pH adjuster. Preferred buffers or pH adjusters include weak acids and bases such glycine/sodium hydroxide, citric acid, lactic acid, succinic acid, acetic salt and salts thereof. Frequently a mixture of buffering system is used such as sodium citrate and citric acid.

Carriers suitable for use with hair care compositions of the present invention include, for example, those used in the formulation of hair sprays, mousses, tonics, waters, creams gels, shampoos, conditioners, and rinses. The choice of appropriate carrier will depend on the particular product to be formulated. The carriers used herein can include a wide range of components conventionally used in hair care compositions. The carriers can contain a solvent to dissolve or disperse the styling compound being used, with water, the C₁-C₆ alcohols, lower alkyl acetate and mixtures thereof being preferred. The carriers can also contain a wide variety of additional materials such as acetone, hydrocarbons (such as isobutane, hexane, decene), halogenated hydrocarbons (such as Freons) and volatile silicones such as cyclomethicone.

When the hair care composition is a hair spray, tonic, gel, or mousse the preferred solvents include water, ethanol, volatile silicone derivatives, and mixtures thereof. The solvents used in such mixtures may be miscible or immiscible with each other. Mousses and aerosol hair sprays can also utilise any of the conventional propellants to deliver the material as a foam (in the case of a mousse) or as a fine, uniform spray (in the case of an aerosol hair spray). Examples of suitable propellants include materials such as trichlorofluoromethane, dichlorodifluoromethane, difluoroethane, dimethylether, propane, n-butane or isobutane. A tonic or hair spray product having a low viscosity may also utilise an emulsifying agent. Examples of suitable emulsifying agents include nonionic, cationic, anionic surfactants, or mixtures thereof. If such an emulsifying agent is used, it is preferably present at a level of from about 0.01% to about 7.5% by weight based on total weight of the composition. The level of propellant can be adjusted as desired but is generally from about 3% to about 30% by weight based on total weight for mousse compositions and from about 15% to about 50% by weight based on total weight for aerosol hair spray compositions.

Hair styling creams or gels also typically contain a structurant or thickener, typically in an amount of from 0.01% to 10% by weight of the total composition.

Suitable spray containers are well known in the art and include conventional, non-aerosol pump sprays i.e., “atomisers”, aerosol containers or cans having propellant, as described above, and also pump aerosol containers utilising compressed air as the propellant.

The formulation may include conditioning materials such as surfactants, cationic conditioners suitable for hair, quaternary silicone polymers, silicone based conditioners and their emulsions, and amino functional silicones and their emulsions.

Further general ingredients suitable for all product forms include, sun-screening agents, anti-dandruff actives, carboxylic acid polymer thickeners for hair shampoo and conditioner compositions and emulsifiers for emulsifying the various carrier components of the compositions of the invention.

The compositions of the present invention may also contain adjuncts suitable for hair care. Generally such ingredients are included individually at a level of up to 2, preferably up to 1 wt % of the total composition. Suitable hair care adjuncts, include amino acids, sugars and ceramides.

The method of the invention comprises applying compositions of the invention to the hair preferably followed by a heating step. The hair should be heated to a temperature above 100° C., preferably above 150° C., more preferably above 180° C.

The invention has the further advantage that successive applications of product according to the invention give enhanced straightening benefits. Preferably, the product of the invention is applied at least twice.

The following non-limiting examples further illustrate the preferred embodiments of the invention. All percentages referred to in the examples and throughout this specification are by weight based on total weight unless otherwise indicated.

### EXAMPLES

Hair was washed and conditioned with US Sunsilk Straighten up shampoo and conditioner. 2 grams of control mousse applied to hair on one side of head and massaged in.

2 grams of prototype mousse applied to hair on the other side of head and massaged in.

The test was conducted blind (i.e. neither stylist nor panellist knows which side of the head is treated with the control and which is treated with the prototype).

After product application the hair was blow dried straight, then ironed straight using GHD straightening irons.

Stylist and panellist assessed the hair post treatment in salon. The panellist then re assessed their own hair following their next normal wash (using their own normal products and regime and at a time of their choosing, typically 1 to 2 days later).

Assessments are scored on a ten point scale, a high score is beneficial. Data was then collated and analysed to produce summary. Significant wins and losses are shown (significance figures are for pair comparison data, e.g. likely hood of scoring prototype higher than control).

TABLE 1

| | | | % active | | | | | |
|---|---|---|---|---|---|---|---|---|
| | | | Example | | | | | |
| Trade name | INCI | Supplier | A | B | C | 1 | 2 | 3 |
| Genamin | behentrimonium | Clariant | 0.3 | 0.3 | 0.3 | 0.3 | 0.3 | 0.3 |
| KDMP | chloride | | | | | | | |
| Cetyl | cetyl alcohol | | 0.3 | 0.3 | 0.3 | 0.3 | 0.3 | 0.3 |
| Alcohol | | | | | | | | |
| Stearyl | stearyl alcohol | | 0.3 | 0.3 | 0.3 | 0.3 | 0.3 | 0.3 |
| Alcohol | | | | | | | | |
| Emalex 710 | laureth 10 | Ikeda | 1.0 | 1.0 | 1.0 | 1.0 | 1.0 | 1.0 |
| | | Corportation | | | | | | |
| Isopropyl | isopropyl | Cognis | 2.0 | 2.0 | 2.0 | 2.0 | 2.0 | 2.0 |
| Myristrate | myristate | | | | | | | |
| Dow | dimethiconol, | Dow Corning | 1.2 | 1.2 | 1.2 | 1.2 | 1.2 | 1.2 |
| Corning 2- | dimethiconol/ | | | | | | | |
| 1787 | silsesquioxane | | | | | | | |
| Emulsion | copolymer, | | | | | | | |
| | sodium c14-16 | | | | | | | |
| | olefin sulfonate, | | | | | | | |
| | trideceth-12 | | | | | | | |
| AP 40 | isobutane | Harp | 8.0 | 8.0 | 8.0 | 8.0 | 8.0 | 8.0 |
| | butane | International | | | | | | |
| | propane | | | | | | | |
| Vitamin E | tocopherol | Sigma | 0 | 0 | 1.0 | 5.0 | 3.0 | 1.0 |
| Vitamin C | ascorbic acid | Sigma | 0 | 10.0 | 0 | 5.0 | 5.0 | 5.0 |

To 100%

TABLE 2

| | Wins/Losses | Wins/Losses |
|---|---|---|
| Prototype | Post Treatment | Post rewash (at home) |
| Example b | Grip, most straight, | Straight Look (90% sig) |
| | least fluff | Ease of style Creation |
| | | (90% sig) |
| Example 1 | Grip, most straight, | Straight looking (95% sig) |
| | least fluff | Good Feel (80% sig) |
| | Straight looking | |
| | (90% sig) | |
| Example 2 | Most straight, | **Straight looking (99% sig)** |
| | least fluff | Satisfaction (90% sig) |
| | Straight looking | Smoothness (80% sig) |
| | (90% sig) | Good Feel (90% sig) |
| | Smoothness | Ease style Creation (90% sig) |
| | (90% sig) | Ease style Creation compared |
| | | to old regime (80% sig) |
| Example 3 | Most straight, | Straight looking (90% sig) |
| | least fluff | |
| | Grip, ease of | |
| | absorption, soft, | |
| | smooth, overall | |
| | quality of style | |
| Example C | Most straight, | No significances |
| | least fluff | |
| | absorption, soft, | |
| | smooth, overall | |
| | quality of style | |
| | Smoothness (>80% sig) | |
| | ease of absorption | |

To demonstrate the incremental benefit of compositions of the invention 3 grams of Example 3 was placed on a mannequin half head, comparative Example A was placed on the other alterative side. After product application the hair was blow dried straight, then ironed straight using GHD straightening irons. The mannequin heads were subjected to 3 sets of 10 agitation shakes with 10 minutes resting period between each set, whilst at 30° C. and 80% relative humidity.

A robotic system photographed the mannequin heads at various angles and data was calculated from average scores given by 12 panellists rating the photographs of the appropriate side of head. The scores were used as comparative for the remaining assessments. The mannequin heads were washed and conditioned with the Sunsilk Straighten Up range and the process described above repeated two times so the hair was treated up to 3 times.

The results in Table 3 describe the scores of hair treated twice or three times with product as compared with hair treated once with product.

TABLE 3

| | Straightness | | Alignment | |
|---|---|---|---|---|
| | Example 3 | Example A | Example 3 | Example A |
| 2 times treated | 8.2 | 5.7 | 20.0 | −6.7 |
| 3 times treated | 46.9 | 7.5 | 62.5 | 13.3 |

The results in table 3 demonstrate that hair tread consecutively with the product of the invention has a marked improvement on hair straightness and alignment.

The following is an example of a cream formulation for use with method of the invention.

TABLE 4

| | | | Example 3 | |
|---|---|---|---|---|
| | | | % as | % as |
| | | | 100% | Received |
| Trade Name | Supplier | INCI Name | % | % |
| Lexamine | Innolex | Stearamidopropyl | 0.50 | 0.50 |
| S-13 | | Dimethylamine | | |
| Hydrenol | Cognis | Cetearyl Alcohol | 1.50 | 1.50 |
| MY/Lanette | | | | |
| S3 | | | | |
| Nipagin M | Clariant | Methylparaben | 0.20 | 0.20 |
| White | Carnation | Paraffinium | 1.00 | 1.00 |
| Mineral | | Liquidium | | |
| Oil SU70 | | | | |
| Wacker | Wacker | Dimethicone (and) | 1.00 | 1.7 |
| Belsil | | Trideceth-5 | | |
| HL999 | | | | |
| Tinovis | Ciba (Now | Dimethylacryamide/ | 0.35 | 0.70 |
| CD | part of | Ethyltrimonium | | |
| | BASF) | Chloride | | |
| | | Methacrylate | | |
| | | Copolymer (and) | | |
| | | Propylene Glycol | | |
| | | Dicaprylate/ | | |
| | | Dicaprate (and) | | |
| | | PPG-1 Trideceth- | | |
| | | 6 (and) C10- | | |
| | | C11 isoparaffin | | |
| Glycerin | “Local | Glycerine | 1.00 | 1.00 |
| | Supplier” | | | |
| Vitamin C | Aldrich | Ascorbic acid | | 5.0 |
| Vitamin E | Aldrich | Tocopherol | up to 5% | 3.0 |

| | |
|---|---|
| water | To 100 |

1. Use of greater than a 1:1 wt ratio of vitamin C to vitamin E to straighten hair the vitamin C and vitamin E being present as at least 2 wt % of the total composition.

2. Use according to claim 1 in which the total level of vitamin C to vitamin E is greater than 5 wt % of the total composition.

3. Use according to claim 1 in which the total level of vitamin C to vitamin E is greater than 3 wt % of the total composition.

4. Use according to claim 3 in which the weight of vitamin C to vitamin E is greater than 3:2.

5. Use according to claim 1 in which the weight of vitamin C to vitamin E is less than 5:1.

6. Use according to claim 1 in which the composition is in the form of a mousse.

7. Use according to claim 1 in which the composition is in the form of a cream.

8. Use according to claim 1 in which the composition further comprises a cationic conditioning agent.

9. Use according to claim 1 in which the composition further comprises a silicone.

10. Use according to claim 1 in which the composition further comprises an alpha-hydroxy acid.

11. Use according to claim 1 in which the composition is packaged so to minimise the products exposure to oxygen.

12. Use according to claim 1 in which hair treated with the composition of the invention is not rinsed or washed for at least one hour.

13. Use according to claim 1 wherein the composition is applied to the hair in successive applications.

14. A method of straightening hair which method comprises the step of applying to the hair a composition comprising at least 2 wt % of the total composition of vitamin C and vitamin E and wherein the ratio of vitamin C to vitamin E is greater than 1:1.
